(19) United States
(12) Patent Application Publication
(10) Pub. No.: US 2006/0280705 A1
(43) Pub. Date: Dec. 14, 2006

(54) Cosmetic preparation

(76) Inventors: Werner Bruechert, Oberasbach (DE); Ursula Hunsinger, Nuernberg (DE); Willy Weiss, Altdorf (DE)

Correspondence Address: BACHMAN & LAPOINTE, P.C., 900 CHAPEL STREET, SUITE 1201, NEW HAVEN, CT, 06510, US

(21) Appl. No.: 10/552,273

(22) PCT Filed: Mar. 30, 2005
(86) PCT No.: PCT/EP05/03330

§ 371 Date: Aug. 21, 2006

(30) Foreign Application Priority Data
Mar. 31, 2004 (EP) 04007812.3
Jun. 4, 2004 (DE) 10 2004 027 282.4

(51) Int. Cl. A61K 8/81; A61K 8/73; A45D 33/24

(52) U.S. Cl. 424/63; 424/70.16; 424/70.17; 132/294

(57) ABSTRACT

A cosmetic preparation which comprises microparticles distributed in an adhesive solution wherein the microparticles contain a substance affording an esthetic effect.

### BACKGROUND OF THE INVENTION

The invention concerns a cosmetic preparation for the application of decorative elements to keratinic material, and a kit which contains said preparation.

The most widely varying substances in micro-encapsulated or nano-encapsulated form are known from the state of the art and are also available as a commercial product. Thus it has long been known for example for active substances encapsulated in nanoparticles to be added to a cream or lotion in order to take the contained substances to their location of action and liberate them there. It is also known to encapsulate delicate and sensitive ingredients such as fragrances which, when the capsule is broken open, release their content. Light protection filter substances, active substances, coloring agents and the like have also already been encapsulated.

Micro-encapsulated substances are available both in the form of dry powder and also in the form of dispersions, preferably in water. The microcapsules usually comprise natural or synthetic polymers which are more or less resistant. In addition it is also known to produce translucent particles with the so-called sol-gel procedure.

It is also known from WO 90/13282 for liquid crystals to be formulated in microcapsule form and then applied dispersed in a carbopol gel as mascara. A disadvantage with a preparation of that kind however is that the viscosity of a carbopol gel is heavily pH-dependent and becomes capable of flow for example at the pH of the skin. On the other hand carbopol is a substance which swells very severely and which shrinks greatly after application, which results in irregular application layers which easily flake off. As carbopol can start to dissolve in particular in the slightly acid state, contact with perspiration can cause the gel to run and the eyelashes to stick together, which is perceived as being unpleasant.

Many effective agents are known which for example have iridescent or glittering properties but which cannot be used in decorative cosmetics as either they are incompatible with the known carrier materials or they themselves represent a health risk. Thus for example metal glitter can get into the tear duct when it is applied to the face or hair and can thereby result in disturbances to the tear discharge flow.

Therefore the object of the invention was to find a possible way of using for example agents which are incompatible with cosmetic carriers or however agents which in free form could represent a health risk, for decorative purposes.

### SUMMARY OF THE INVENTION

The object is attained in that microparticles are provided in an adhesive solution, which are suitable for the application of decorative elements to keratinic material. The subject-matter of the invention is therefore a preparation for the application of decorative elements to keratinic material containing microparticles in an adhesive solution.

### DETAILED DESCRIPTION

A preparation was developed, which combines various advantages. In accordance with the invention it is now possible to use agents which are incompatible but highly decorative in cosmetics. The preparation can be easily applied and, if desired, also easily removed again. Many attractive, surprising decorative effects which were hitherto not known in that form can be achieved and also combined.

What is essential for the present invention is the provision of microparticles containing substances which afford an esthetic effect. A further essential feature is that the microparticles are in an adhesive solution.

By virtue of their properties the microparticles according to the invention can be applied to keratinic material and remain there as long as is desired. In that respect the term keratinic material is used to denote skin, mucous membrane, semi-mucous membrane and hair. Use is considered in particular in relation to eyelashes, eyebrows, facial hair and beards and hair at the temples. Equally it is possible for the preparation to be applied to strands of hair or individual regions of the shock of hair, for example hair which falls over the forehead. A further possibility involves applying the preparation to lids, rims of the eyes, the sides of the nose, ear lobes, décolletage or other parts of the body on which a decorative effect is to be achieved. Preferably the preparation according to the invention is used for decorating eyelashes. The preparation can possibly also be applied to eyelashes coated with mascara.

An essential constituent of the preparation according to the invention is microparticles containing a means producing an esthetic impression, hereinafter also referred to as an agent. The term microparticles is used in accordance with the present invention to denote both microcapsules and also microspheres and liposomes. Microcapsules are particles which have a core with agent which is enclosed by a wall. Microspheres are particles comprising a matrix in which the agent is embedded. Microspheres generally have pores. Liposomes are particles which are made up of lipid-like molecules. In a preferred embodiment the polymer or copolymer forming the wall material or the matrix is a transparent or semi-transparent material and the agent affording an esthetic effect is arranged internally in the particle. It is however also possible for the agent affording the esthetic effect to be adsorbed on the microspheres or absorbed, for example in the outwardly disposed pores thereof. In this embodiment it is then preferred for the microsphere also to be covered by a thin layer of a transparent plastic material so that the agent cannot escape.

The material for wall, matrix or liposome wall, also referred to hereinafter as the carrier material, is a natural or synthetic polymer or copolymer or a silicate produced with the sol-gel procedure. Besides in the embodiment in which the agent is absorbed or adsorbed externally on the particle, the carrier material is transparent or semi-transparent in order to allow the agent to be visible. It is advantageous if the particles are as elastic as possible and on the other hand withstand a certain pressure without bursting open. If the microparticles are applied in the vicinity of the eye and get into the eye, they should not act like a hard foreign body and they should not irritate the eye. Therefore, biocompatible, preferably elastic or soft polymers and copolymers are suitable for the production of the microparticles, in a particularly preferred feature natural polymers such as gelatin, alginates, cellulose, guar gum, xanthan gum, agar agar, gum arabic or derivatives thereof and mixtures thereof, in particular highly cross-linked derivatives which have advantageous properties in regard to water resistance. It is also possible to use mixtures of the specified substances.

If a coating layer is used, a transparent, film-forming material which is compatible with the agent and the microparticles is appropriately used for that purpose, preferably a polymer or copolymer as above or as defined hereinafter for the adhesive layer.

The polymer selected for the carrier material and possibly the coating layer is so adapted in a preferred embodiment that it is not dissolved or broken down either by pure water or by tear fluid, perspiration or skin grease in a temperature range of up to 40° C., preferably 50° C. In addition the carrier material must be inert in relation to the material to be encapsulated, that is to say it is not to either react with or alter the agent.

The microparticles should be of a size in the range of between 500 nm and 2 mm. Particles of a mean diameter of below 500 nm are generally too small to be able still to afford an effect. Particles of more than 2 mm can be perceived as foreign bodies in particular when applied to the eyelashes.

As carbopol can begin to dissolve in particular in the slightly acid state, it can result in the eyelashes sticking together, which is perceived as being unpleasant.

Preferably microparticles are used of a mean diameter of between 15 μm and 1.5 mm and in particular in a range of sizes of between 0.2 mm and 1 mm. Generally the microparticles are spherical, preferably ball-shaped. It is however also possible to consider other shapes such as drop-like, oval or shapes which are rounded in some other fashion.

Production of the microparticles is known per se and does not have to be described in greater detail here. Microparticles are commercially available in the most widely varying sizes and configurations. Production of the microparticles which are filled in accordance with the invention can be effected similarly with processes known to the man skilled in the art.

The microparticles contain a means or agent which causes an esthetic impression, in particular such an agent which is either not compatible with conventional cosmetic agents or which cannot deploy its effect when it is incorporated into agents of that kind. In particular liquid crystals or a mixture of liquid crystals, luminescent, fluorescent, phosphorescent, iridescent, mother-of-pearl-like, thermochromic substances, neon pigments, luminous pigments, interference pigments, metal flakes or spangles, holographic elements, pearlescent agents, inorganic pigments, organic pigments, lakes of soluble organic dyes and/or preparations with UV-active dyes.

It is possible to achieve many different effects with those materials. In accordance with the invention it is possible to use both only one kind of microparticles and also mixtures thereof, in which case individual microparticles can contain mixtures of those agents, but it is also possible for various microparticles with respectively different fillings to be used mixed. In a further embodiment various populations of microparticles can form the preparation, in which case the individual populations can comprise for example identically filled microparticles of the same size, identically filled microparticles of different sizes, microparticles filled with mixtures, microparticles which contain an agent in various colors or configurations, and so forth. Many different configurations and combinations are possible. Thus for example UV-active dyes can be combined with other effect agents in order to produce an effect both in daylight and/or under artificial light and also with black light.

It is possible to provide various microparticles in a preparation so that the male/female user can select the appropriate microparticles for any desired occasion. In a further embodiment microparticles of various sizes each with the same filling or with different fillings can be provided, in which respect the microparticles can involve a wide size distribution or a bimodal or trimodal size distribution. In a preferred embodiment microparticles are loaded with liquid crystals which have an incomparable iridescent effect. In a further embodiment of the invention metal flakes or “spangles” are incorporated into the microparticles. That prevents small metal particles which could block up the tear duct from getting into the eye.

Production of microparticles in various sizes and size distributions can be effected in per se known manner. The size and dispersity can be adjusted by the selection of the process parameters, the tensides and emulsifiers used, agitators and agitator speeds and so forth. All those measures are known to the man skilled in the art and do not need to be described in greater detail here. Preferably the microparticles have a polydispersity in the range of between 1 and 15.

The microparticles are to be brought to the appropriate location and remain clinging there as long as is desired. For that purpose the preparation according to the invention includes, as its second essential constituent, an adhesive solution, preferably an aqueous-based adhesive solution. The adhesive solution used in accordance with the invention contains a cosmetically acceptable adhesive based on suitable polymers and copolymers. It is essential that the adhesive solution is compatible both with skin or hair and also with the microparticles. Thus, the fluid medium is not to alter and not dissolve the microparticles, not even when prolonged storage is involved and not even at temperatures of up to 50° C. Preferably the adhesive is a polymer or copolymer formed from the monomers vinylalcohol, vinylpyrrolidone, acrylates, methacrylates, urethanes, carboxylic acids and alcohols or mixtures thereof. In particular the adhesive is selected from polyvinylalcohol, polyvinylpyrrolidone, polyacrylates, polymethacrylates, polyacrylamides and polymethacrylamides, polyurethanes, polyesterurethanes, and mixtures thereof. Particularly preferably polyvinylacetate, polyvinylpyrrolidone or a mixture of polyvinylalcohol and polyvinylpyrrolidone in an aqueous medium is used as the adhesive. The polymers or copolymers are present dissolved or dispersed in an aqueous medium. The aqueous medium can be water or a mixture of alcohols or biocompatible solvents with water. After application the medium evaporates and the adhesive which remains behind affords adhesion for the particle at the applied location.

The selected adhesive should be such that, after hardening or drying, it withstands the normal skin moisture, perspiration and tear fluid, but can be easily caused to dissolve by warm water at a temperature of more than 40° C., preferably between 40 and 45° C., possibly in the presence of surface-active agents, so that in due course it can be easily removed again from the location at which it was applied.

It will be noted that those polymers and copolymers whose volume swells up to more than five times when they are in an aqueous environment are less suited for the adhesive of the preparation according to the invention. Therefore it is preferred to use a polymer or copolymer or a polymer/copolymer mixture as the adhesive, which in water increases in volume by not more than five times, preferably not more than three times. In a preferred embodiment the adhesive used is a pseudolatex which practically does not swell in an aqueous environment but essentially retains its volume. Pseudolatexes are well known in the area of cosmetics and are familiar to the man skilled in the art.

In a preferred embodiment the density of the adhesive solution is so adjusted that the microparticles float or are suspended in the solution and do not settle. For that purpose the dynamic viscosity of the preparation is adjusted in a range of between 1 and 10,00 mPa·s, preferably between 1 and 3,500 mPa·s, particularly preferably between 1 and 1,000 mPa·s. Particularly good results are achieved with a viscosity in the range of between 1 and 250 mPa·s. In addition the viscosity of the adhesive solution is so adjusted that on the one hand on being taken out the solution does not drip while on the other hand upon being taken out it remains clinging to the microparticles sufficiently to exert an adhesive effect. Preferably an adhesive solution whose viscosity substantially does not change in dependence on the pH is selected.

The microparticles and the adhesive solution are mixed to produce the preparation according to the invention. If the microparticles were obtained in the form of an aqueous dispersion, they are separated prior to mixing from the aqueous medium, for example by sieving.

The pH value of the preparation according to the invention should be so adjusted that on the one hand the properties of the adhesive and microparticles are not adversely affected and on the other hand skin and eyes are not irritated. Preferably therefore the pH value is adjusted in a range of between 5 and 8, particularly preferably between 5.5 and 7.4, which corresponds to the range between the normal pH of a healthy skin and the pH of the tear fluid. In the event that the preparation according to the invention is used in the region of the eye, the pH value should be selected to be rather in the region of the tear fluid, that is to say in a range of between 7 and 7.5, while when application to the skin is involved the pH value should rather be adjusted to be in the lower range. Means for adjusting the pH value are known per se and familiar to the man skilled in the art.

A further subject-matter of the invention is a kit which includes a preparation according to the invention in a container or vessel, as is used in the area of cosmetics. Suitable containers are for example pots, bottles, phials, tubes, bottles with inserted application element, automatic application devices which are offered for example under the brand “Visko-Magic”. An application element of that kind is described for example in DE 198 58 410. This involves a device for applying a liquid, pasty or gel-like product having a storage means for the product, a support connected to the storage means by way of at least one feed passage and an applicator element provided with a plurality of through passages for the product, the outside surface of the applicator element forming an applicator surface which in a preferred embodiment is flocked. Further devices which are also suitable for applying the preparation according to the invention are described in DE 202 04 111 and DE 203 10 777. In the case of those applicator elements the feed passages can be such that the microparticles are individually discharged therefrom. Preferably the preparation according to the invention is introduced into at least partially transparent containers in order to present the content and to demonstrate the esthetic effect involved.

In a preferred embodiment the preparation according to the invention is filled in a container provided with an application element which permits specific and targeted application of individual microspheres from the preparation. Preferably therefore the kit according to the invention is in the form of a bottle or tube provided with an application element in the form of a small tube member, wherein the small tube member is of such a diameter that there is only ever one microparticle that can issue from the front opening, that microparticle being surrounded by adhesive solution. That kit allows the individual microparticles to be applied specifically at the desired location without for example completely coating eyelashes or hair with microparticles. The kit according to the invention therefore makes it possible for for example only one microparticle or only a few microparticles to be put on each eyelash at the tip. As the preparation according to the invention allows microparticles also to be applied to eyelashes which have been previously treated with mascara, it is thus possible for the eyelashes firstly to be colored with mascara in a desired color and then for further effects to be produced with the microparticles at a few locations, for example the tips of the eyelashes.

As stated above the preparation according to the invention can be applied to skin and hair. For that purpose there is appropriately provided an application element which is suitable for the application operation. Preferably suction pipettes, brushes, loops, small hook members, small brush members and the like are used for the application procedure. In that case the microparticles can be placed individually, for example with a suction pipette or a loop, at the respectively desired location. It is also possible for a plurality of microparticles to be simultaneously applied at the desired location, for example with a brush. For the application operation the application element is dipped into the preparation of adhesive solution and microparticles and either a microparticle or a proportion of solution which contains a plurality of microparticles is removed and placed at the desired location. In a further embodiment the microparticles are individually urged out of a pipette and applied. The pipette can be a suction pipette with which the preparation was previously sucked up out of a container. In a further embodiment the preparation according to the invention is contained in a container in tube form, in which case the tube container has an applicator in the form of a small tube member at one end. The balls are then urged out of the tube into the tube-like applicator and applied by way thereof at the desired location.

When the microparticles are removed from the container the microparticles are enclosed by the adhesive solution. The viscosity of the solution is preferably such that on the one hand, on being taken out, the solution does not drip off and on the other hand adhesive clings sufficiently to the microparticles that a good adhesive action is achieved. The particles surrounded by adhesive solution are then brought to the appropriate location and adhere there. Preferably the adhesive solution is such that after application it dries quickly and thereafter is so longer sticky.

In order further to describe the invention some examples of compositions will be set forth hereinafter. All details in the Table are in % by weight and relate to the total amount of the mixture:

| | Example 1 | Example 2 | Example 3 | Example 4 | Example 5 |
|---|---|---|---|---|---|
| Water, | 71.750 | 70.250 | 68.750 | 70.250 | 68.750 |
| dist. | | | | | |
| PVA, high- | 12.500 | 7.500 | 15.500 | 7.500 | 2.500 |
| mol. | | | | | |
| PVA, | — | 6.500 | — | — | 7.500 |
| medium- | | | | | |
| mol. | | | | | |
| Glycerin | 11.000 | 11.000 | 11.000 | 11.000 | 11.000 |
| Butane | 4.500 | 4.500 | 4.500 | 4.500 | 4.500 |
| diol | | | | | |
| Preserving | 0.250 | 0.250 | 0.250 | 0.250 | 0.250 |
| agent | | | | | |
| Soda lye | q.s. | q.s. | q.s. | q.s. | q.s. |
| 10% | | | | | |

For the production operation the water is presented in a suitable vessel and the polymer powder dispersed therein by means of a propeller agitator. Thereupon glycerin, butane diol and the preserving agent are added. The pH value is then adjusted to the desired value by the addition of soda lye. Commercially available microcapsules which are available in the form of an aqueous dispersion are separated from the dispersing agent by suitable measures such as filtration through a glass frit or the like or centrifuging and immediately added to the above-described adhesive mixture with moderate agitation with the propeller agitator. The finished mixture contains between 0.1 and 50% by weight, preferably between 3 and 30% by weight and particularly preferably between 5 and 25% by weight of microcapsules, in dependence on the intended optical effect.

1-30. (canceled)

31. A cosmetic preparation for the application of decorative elements to keratinic material comprising microparticles in an adhesive solution, wherein the microparticles contain the decorative elements.

32. A cosmetic preparation as set forth in claim 31, wherein the keratinic material is hair or skin.

33. A cosmetic preparation kit as set forth in claim 31, wherein the keratinic material eyelashes, eyebrows, facial hair and temple hair.

34. A cosmetic preparation as set forth in claim 31, wherein the microparticles are selected from the group consisting of microcapsules, microspheres, liposomes and mixtures thereof.

35. A cosmetic preparation as set forth in claim 31, wherein the microparticles comprise a transparent or semi-transparent case and a material affording an esthetic impression as core material.

36. A cosmetic preparation as set forth in one claim 35, wherein the material affording an esthetic impression are selected from the group consisting of liquid crystals, luminescent, fluorescent, phosphorescent, iridescent, mother-of-pearl-like, thermochromic substances, neon pigments, luminous pigments, interference pigments, metal flakes or spangles, holographic elements, pearlescent agents, inorganic pigments, organic pigments, lakes of soluble organic dyes, preparations with UV-active dyes and mixtures thereof.

37. A cosmetic preparation as set forth in claim 31, wherein the microparticles comprise elastic microspheres which have substances affording an esthetic impression on their surface and/or in the pores.

38. A cosmetic preparation as set forth in claim 37, wherein the microspheres are coated with a material affording an esthetic impression and are additionally covered with a transparent film.

39. A cosmetic preparation as set forth in claim 31, further including a carrier material for the microparticles which comprises a material selected from the group consisting of natural polymer, synthetic polymers, a silicate produced by a sol-gel procedure, and mixtures thereof.

40. A cosmetic preparation as set forth in claim 39, wherein the polymers are selected from the group consisting of gelatin, alginates, cellulose, guar gum, xanthan gum, agar agar, gum arabic, a highly cross-linked derivative of one of the foregoing polymers, and mixtures thereof.

41. A cosmetic preparation as set forth in claim 39, wherein the carrier material of the microparticle comprises a polymer or copolymer which is not dissolved by water, tear fluid and skin grease up to a temperature of 50° C.

42. A cosmetic preparation as set forth in claim 31, wherein the adhesive solution comprises an aqueous or aqueous/alcohol solution of an adhesive.

43. A cosmetic preparation as set forth in claim 31, wherein the adhesive comprises polymers and/or copolymers which after hardening are not dissolved or caused to swell by water at a temperature below 40° C. but which can be caused to dissolve by water at a temperature of greater than 40° C.

44. A cosmetic preparation as set forth in claim 31, wherein the adhesive comprises a polymer or copolymer selected from the group consisting of polyvinylalcohol, polyvinylpyrrolidone, PVA/PVP, acrylate, methacrylate, urethane, polyester, polyesterurethane, and mixtures thereof.

45. A cosmetic preparation as set forth in claim 31, wherein the adhesive solution has a viscosity which is substantially pH-independent.

46. A cosmetic preparation as set forth in claim 31, including a dye solution having density such that the microparticles float in the solution surrounding them.

47. A cosmetic preparation as set forth in claim 31, wherein the microparticles are spherical or drop-shaped.

48. A cosmetic preparation as set forth in claim 31, wherein the microparticles have a mean diameter of between 500 nm and 2 mm.

49. A cosmetic preparation as set forth in claim 31, wherein the microparticles have a mean diameter of between 15 μm and 1.5 mm.

50. A cosmetic preparation as set forth in claim 31, wherein the microparticles have a polydispersity in a range of between 1 and 15.

51. A cosmetic preparation as set forth in claim 31, wherein the microparticles are filled with different substances producing the esthetic impression.

52. A cosmetic preparation as set forth in claim 31, wherein the microparticles contain different colorings.

53. A cosmetic preparation as set forth in claim 31, including a plurality of microparticle populations, wherein at least one population has color effects and at least one population has iridescent or glittering effects.

54. A cosmetic preparation as set forth in claim 31, including at least two populations of microparticles, wherein within the individual populations the size is in a narrow range of +/−10 μm.

55. A cosmetic preparation as set forth in claim 31, wherein the preparation has a dynamic viscosity in a range of between 1 and 10,000 mPa·s.

56. A cosmetic preparation as set forth in claim 31, wherein the preparation has a dynamic viscosity in a range of between 1 and 3,500 mPa·s.

57. A kit containing a cosmetic preparation as set forth in claim 31, wherein the preparation is a container.

58. A kit as set forth in claim 57, further comprising an applicator for applying the preparation.

59. A kit as set forth in claim 58, wherein the applicator is an application element selected from the group consisting of suction pipettes, brushes, loops, small hook members, small brush members and tweezers.

60. A kit as set forth in claim 58, wherein the container comprises a bottle or a tube, and the applicator comprises a small tube member which is fitted thereon.

61. A kit as set forth in claim 60, wherein the tube member has a front opening diameter wherein only one microparticle is adhesive solution can issue from the front opening at a time.
